(19) United States
(12) Patent Application Publication
(10) Pub. No.: US 2012/0310118 A1
(43) Pub. Date: Dec. 6, 2012

(54) SHOULDER TORQUE AND RANGE OF MOTION DEVICE

(76) Inventors: Joseph J. Sarver, Abington, PA (US); Louis J. Soslowsky, Penn Valley, PA (US)

(21) Appl. No.: 13/577,589

(22) PCT Filed: Feb. 8, 2011
(86) PCT No.: PCT/US11/24022

§ 371 Date: Aug. 7, 2012

Related U.S. Application Data
(60) Provisional application No. 61/302,375, filed on Feb. 8, 2010.

(51) Int. Cl. A61B 5/11

(52) U.S. Cl. 600/595

(57) ABSTRACT

The invention relates to a device for measuring shoulder stiffness and range of motion. Specifically, the invention relates to a device for measuring shoulder stiffness and range of motion in human subjects, and methods for making and using thereof.

### CROSS-REFERENCE TO RELATED APPLICATIONS

This application claims priority to U.S. Provisional Patent Application 61/302,375, filed Feb. 8, 2010, which is incorporated by reference herein in its entirety.

### FIELD OF THE INVENTION

The invention relates to a device for measuring shoulder stiffness and range of motion. Specifically, the invention relates to a device for measuring shoulder stiffness and range of motion in human subjects, and methods for for making and using thereof.

### BACKGROUND OF THE INVENTION

The rotator cuff consists of four muscle tendon units that constrain the humeral head (ball of shoulder joint) onto the glenoid of the scapula (socket of the shoulder joint) during arm motion, as well as, contribute to motion. Rotator cuff abnormalities result in pain and lost shoulder function and are typically treated with immobilization, surgery, or a combination of the two.

Following surgical repair, shoulder motion is restricted in order to promote healing. However, it should be mobilized enough to ensure that the healing pattern doesn't interfere with the normal range of motion. Physicians currently use various scoring systems such as one developed by the American Shoulder and Elbow Surgeons (ASES), which separates the assessment of shoulder functionality into “objective” and “subjective” categories.

Clinicians would like to find the maximum amount of time to restrict shoulder motion without sacrificing range of motion (e.g. shoulder stiffness). Presently, clinicians manually move the shoulder joint and use their judgment in assessing the range of motion. Thus, physicians are not able to quantitatively compare the degree of motion of the joint prior to and after repair surgery.

While there are limited means for assessing repair integrity clinically (ultrasound, or MRI), currently there does not exist a device that quantitatively measures shoulder stiffness by evaluating the degree of motion and the amount of torque that the patient is able to exert. Other currently used method involves physical tests and physicians' subjective judgments on mobility and stiffness.

Accordingly, there exists a need for a clinical device that quantitatively measures rotational stiffness as well as the range of motion of a subject's shoulder.

### SUMMARY OF THE INVENTION

In one embodiment, provided herein is a device for measuring shoulder stiffness in human subjects. In another embodiment, the device comprises an adjustable armature that is capable of being secured to a forearm of a human subject. In another embodiment, the armature is mounted on a stand. In another embodiment, the stand rests on locking caster wheels constructed of extruded aluminum and adjusted from about 70 to about 140 cm in height to account for more than about 95% of the adult human population. In another embodiment, the device comprises a sensor assembly comprising a torque load cell, two ball bearing joints, and an orientation sensor, wherein, in another embodiment, the torque load cell is mounted below the armature, in-line with a rigid axle, passing through the ball bearing joints, further wherein the orientation sensor is disposed on the undersurface of the axle, and, in another embodiment, wherein the device is capable of measuring torque as the forearm of the human subject is rotated, from which shoulder stiffness and range of motion can be computed.

In another embodiment, provided herein is a method for making a device for measuring shoulder stiffness and range of motion in a human subject. In another embodiment, the method involves joining an adjustable armature to a portable stand. In another embodiment, the armature is secured to the subject's forearm, near the elbow, with Velcro straps. In another embodiment, the method involves resting the stand on locking caster wheels, that are, in another embodiment, constructed of any suitable material known in the art, such as, but not limited to extruded aluminum. In another embodiment, the stand is adjusted from about 70 to about 140 cm in height to account for >95% of the adult population. In another embodiment, the method involves mounting a six-degree of freedom load cell below the armature and doing so in-line with a rigid axle which passes through a low-friction roller bearing joint. In another embodiment, an orientation sensor is placed on the undersurface of the axle, as well as a handle from which the operator rotates the subject's arm in either internal or external directions. In another embodiment, the device has at least one handle. In another embodiment, the operator passively rotates the subject's arm in either internal or external directions. In another embodiment, an orientation sensor is be placed on the undersurface of the axle, as well as a handle from which the operator will rotate the subject's arm in either internal or external directions. In another embodiment, the method involves connecting the the sensors of the device using connecting modules to a processing machine, for processing measured data. In another embodiment, the method involves processing the data using the processing machine and a suitable analytic software available in the art and visualizing the results using a visual display or monitor that, in another embodiment, is mounted on the device for displaying torque and angle displacement measurements.

In one embodiment, provided herein is a method for accurately quantifying shoulder joint stiffness comprising using the device provided herein.

In another embodiment, provided herein a method of measuring joint stiffness in the shoulder of a human subject comprising the steps of placing the device provided herein on either side of said human subject's upper extremity and measuring a set of parameters as the upper extremity is externally and internally rotated by an operator. In another embodiment, the upper extremity is rotated passively.

In one embodiment, provided herein is a method of monitoring the progress of a human subject's rehabilitation following a procedure in which increased shoulder stiffness and decreased range of motion are of concern, comprising the step of using the device provided herein.

In another embodiment, provided herein a method of assessing the effect of immobilization on repair integrity, shoulder stiffness and range of motion following rotator cuff repair surgery in a human subject, the method comprising the step of using the device provided herein.

In one embodiment, provided herein is a method of measuring passive shoulder mechanics before and at various pre-determined time points following treatment, the method comprising the step of using the device provided herein. In another embodiment, it is to be understood that a skilled artisan is able to devise a set of time points as required for each particular subject or pool of subjects.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which

FIG. 1 is a schematic representation of a device for measuring shoulder stiffness and range of motion, according to one embodiment of the invention;

FIG. 2 is a photographic representation of the device, according to one embodiment of the invention;

FIG. 3 is a photographic representation of the armature, sensor assembly, and handle of the device, according to one embodiment of the invention;

FIG. 4 shows measuring torsional stiffness of a subject's shoulder, according to one embodiment of the invention; and

FIG. 5 shows an example torque-angular displacement data from a rat shoulder in external (blue) and internal (red) with key measurements highlighted, according to one embodiment of the invention.

### DETAILED DESCRIPTION OF THE INVENTION

The invention relates to a device 10 for measuring shoulder stiffness and range of motion. Specifically, the invention relates to a device 10 for measuring shoulder stiffness and range of motion in human subjects, and methods for for making and using thereof.

In one embodiment, provided herein is a device 10 for measuring shoulder stiffness and range of motion in a human subject, the device comprising an adjustable armature 14 that is capable of being secured to a forearm of a human subject; a sensor assembly 42 comprising a torque load cell 26, a ball bearing joint 30, and an orientation sensor 34. In another embodiment, the torque cell 26 is mounted below said armature 14, in-line with a rigid axle 38, and is capable of passing through said ball bearing joints 30. In another embodiment, the device has a minimum of one ball bearing joint 30. In another embodmient, the orientation sensor 34 disposed on the undersurface of said axle 38. In another embodiment, the device 10 is capable of measuring shoulder stiffness and range of motion during passive rotation of the forearm of a human subject.

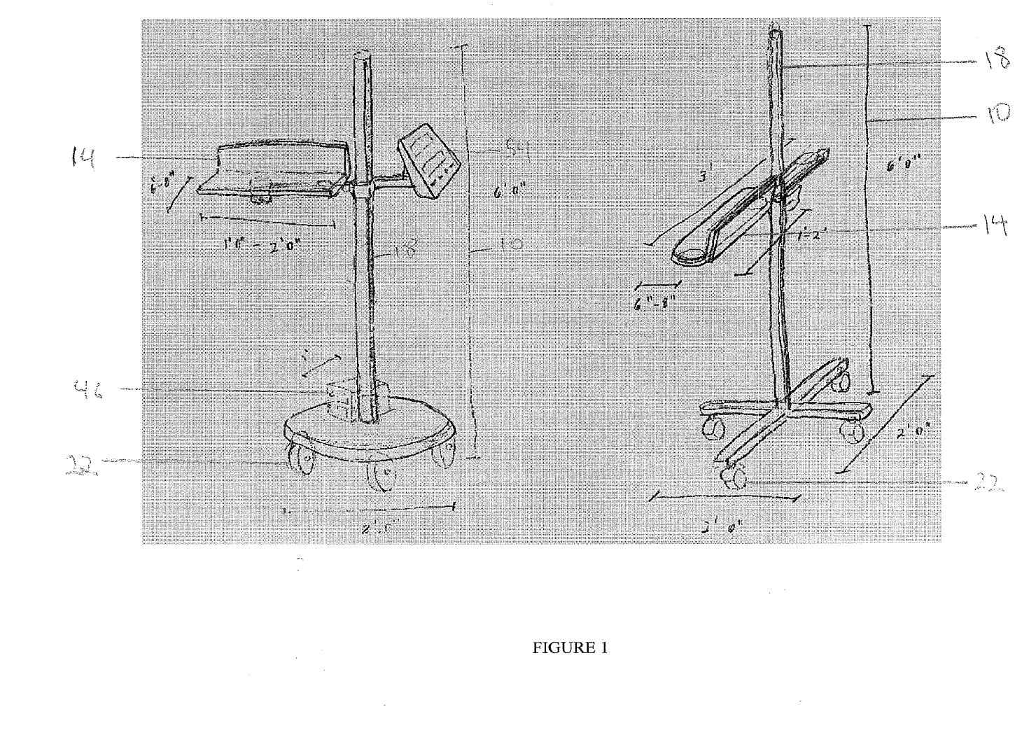

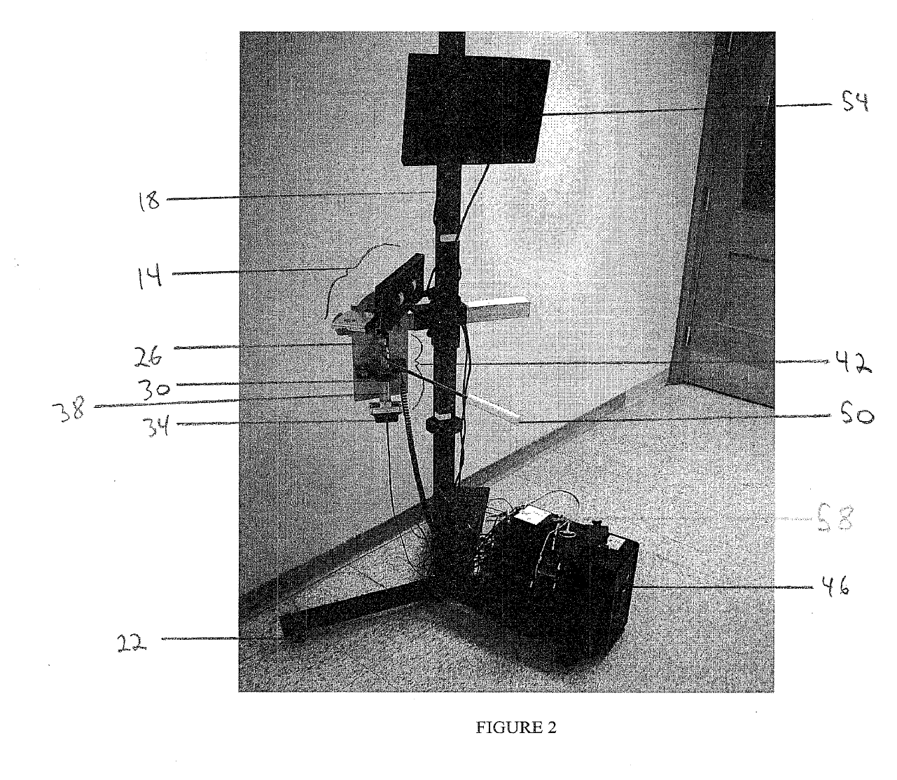

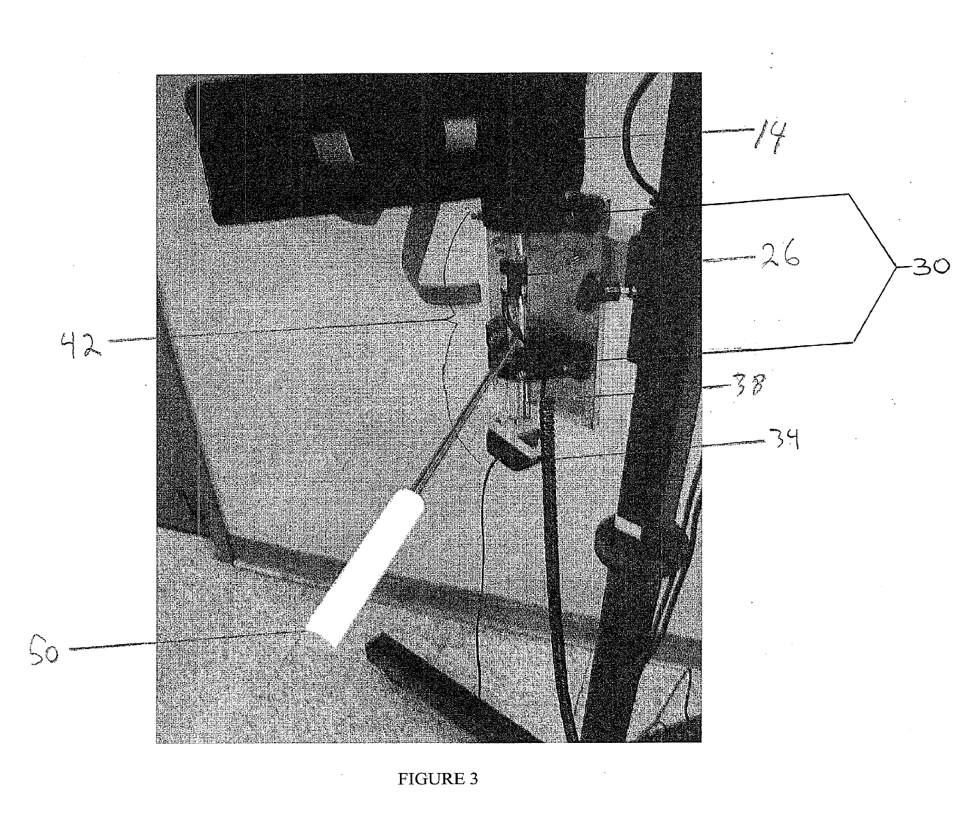

Referring to the drawings and the characters of reference marked thereon, FIGS. 1, 2 and 3 illustrate an embodiment of the device designated generally as 10.

In one embodiment, the device 10 for measuring shoulder stiffness and range of motion in a human subject is made of an adjustable armature 14, a sensor assembly 42, a processing machine 46, and a visual display or monitor 54. In another embodiment, the processing machine 46 comprises a torque cell amplifier 58. In another embodiment, the processing machine comprises a data acquisition card.

In another embodiment, the device 10 for measuring shoulder stiffness and range of motion in a human subject is made of an adjustable armature 14 that is capable of being secured to a forearm of a human subject. In another embodiment, the human subject is seated. In another embodiment, the human subject is standing. In another embodiment, the armature 14 adjusts in height. In another embodiment, the armature 14 is made of soft non-porous material that is disinfectable and enables comfortability for a human subject. In another embodiment, the armature 14 is easily removed to switch between left and right extremity. In another embodiment, the armature is mounted on a stand 18. In another embodiment, the sensor assembly including a six-degree of freedom torque load cell 26 is mounted below the armature 14 and is in-line with a rigid axle 38. In another embodiment, it is to be understood that any operative torque load cell of various degrees of freedom can be used to make the device 10 provided herein. In another embodiment, the sensor assembly 42 also includes an orientation sensor 34 that is placed on the undersurface of the axle, as well as a handle 50 from which the operator can passively rotate the subject's arm in either internal or external directions. In one embodiment, the handle 50 is removable. In another embodiment, the handle 50 is used for applying torque. It is to be understood that the orientation sensor used to make the device 10 is not limited to the orientation sensor disclosed herein, instead, any operative orientation sensor known in the art can be used. In another embodiment, the device 10 has connecting modules for connecting the sensors 26, 34 to a processing machine 46, for processing measured data. In another embodiment, the data processing machine 46 collects, stores, and processes the measured data and displays it in a visual display or monitor 54 that, in another embodiment, is mounted on the device 10 for displaying torque and angle displacement measurements.

In one embodiment, the terms “torque sensor” or “torque load cell” are synonymous in that they each refer to the same component.

In one embodiment, the device 10 is calibrated for optimum efficiency in making measurements. In another embodiment, the device 10 is capable of being calibrated to provide accuracy or precision in measuring rotational stiffness. In another embodiment, the device 10 is calibrated to provide accuracy or precision in measuring rotational stiffness. In another embodiment, the calibration object used is one with known stiffness, such that accuracy and precision of rotational stiffness can be determined In another embodiment, the calibration object is a steel spring or any other object/material with known stiffness, as will be understood by a skilled artisan.

In one embodiment, the portable stand 18 to which the armature 14 is connected to, rests on locking caster wheels 22 constructed of extruded aluminum and adjusted from about 70 to about 140 cm in height to account for more than 95% of the adult human population. In another embodiment, the armature 14 is made out of foam padding/molded plastic and is secured near the subject's elbow, with velcro straps. In another embodiment, the armature 14 of the device 10 is a pivoting armature that allows internal/external rotation by an operator and, in another embodiment, has the capability to rotate around a main vertical pole.

In another embodiment, the torque load cell 26 passes through two low-friction roller bearing joints 30. In another embodiment, the device has a minimum of one ball bearing joint 30. In another embodiment, the device 10 has two or more ball bearing joints 30. In another embodiment, the torque load cell 26 passes through a minimum of one ball bearing joint 30. In another embodiment, the torque load cell 26 passes through two or more ball bearing joints 30.

In one embodiment, the device 10 has dimensions of 28 inches long by 6 inches wide. In another embodiment, the device has a skeleton made of any material as will be understood by a skilled artisan, including, but not limited to aluminum. In yet another embodiment, the skeleton is able to withstand at least a 3-foot drop. It is to be understood that variations to the dimensions or alterations to the components of the device 10 provided herein e.g., wherein the device 10 is altered to comprise one or more sensors, handles, armatures, etc. are also encompassed within the invention provided herein.

In one embodiment, the device 10 is capable of measuring torque and angular displacement as the upper extremity is externally and internally rotated. In another embodiment, the subject is seated, and the device is positioned such that the subject's arm is fully adducted with zero degrees of flexion. In another embodiment, the device 10 incorporates the length of a patient's arm, which current testing for shoulder stiffness and range of motion does not account for.

In another embodiment, the device 10 measures shoulder stiffness and range of motion during passive rotation of a human subject's forearm by making use of its sensor assembly. In another embodiment, the sensor assembly 42 includes a six-degree of freedom torque load cell 26 (mounted below the armature), two ball bearing joints 30, and an orientation sensor 34. In another embodiment, the torque load cell 26, ball bearing joints 30 and orientation sensor 34 are in-line with a rigid axle 38, and passes through the ball bearing joint 30, wherein the ball bearing joints are low-friction roller bearing joints. In another embodiment, the orientation sensor 34 is disposed on the undersurface of the axle 38. Further, and in another embodiment, the torque load cell 26 has a maximum torque of about 100 Nm. In another embodiment, the torque sensor is any torque sensor known in the art. In another embodiment, the torque sensor is an inline rotary torque sensors that is used in line with rotating shafts. In another embodiment, rotary torque sensors are strain gage type that measure values from about 0.5 NM to 1000 NM. In another embodiment, the torque sensor/cell 26 is utilized with the orientation sensor 34 to measure force applied in an angular motion. In one embodiment, the orientation sensor 34 is fully temperature compensated, is calibrated for misalignment and gyro G-sensitivity, and supports hard iron field calibration. In another embodiment, the orientation sensor 34 produces outputs in formats of Euler angles, quaternions and orientation matrices. A great benefit to this powerful tool is its small size, light weight, and low power usage. In another embodiment the orientation sensor 34 is any orientation sensor known in the art. In another embodiment, the orientation sensor is a microstrain 3DM-GX1® orientation sensor. In one embodiment, the orientation sensor 34 combines three angular rate gyros with three orthogonal DC accelerometers, three orthogonal magnetometers, and embedded microcontroller to output is its orientation in dynamic and static environments. In one embodiment, this sensor 34 is used to examine one range of motion. In another embodiment, the range of motion measured by the sensor 34 is the angular motion. In one embodiment, any software and algorithms known in the art to be useful for analyzing output measurements provided by the two sensors is used for such purposes. In another embodiment, to analyze measurements provided by the two sensors, LabView and customized algorithms are used to study the outputs.

In one embodiment, the device 10 measures torque and angular displacement as the human subject's arm is passively rotated in an internal or external direction by an operator using a handle 48 comprised by the rigid axle 38. In another embodiment, the subject is effectively diagnosed as having unilateral shoulder stiffness and range of motion. In another embodiment, the subject is a healthy individual. In another embodiment, healthy human subjects with a range of different heights, weights, and ages are used to test the feasibility and adjustability of the device.

In one embodiment, the device 10 is routinely calibrated such that accuracy and precision of rotational stiffness is determined. In another embodiment, the device 10 measures a set of parameters as the subject's arm is passively rotated by an operator. In another embodiment, the parameters are the number of internal/external cycles and the speed of rotation. In another embodiment, the parameters enable measuring the range of motion, the peak torque, and the rotational stiffness.

In one embodiment, testing parameters used to arrive at measurements of range of motion, peak torque, and rotational stiffness include the number of internal/external cycles and the speed of rotation. In another embodiment, the operator is a clinician or a physician.

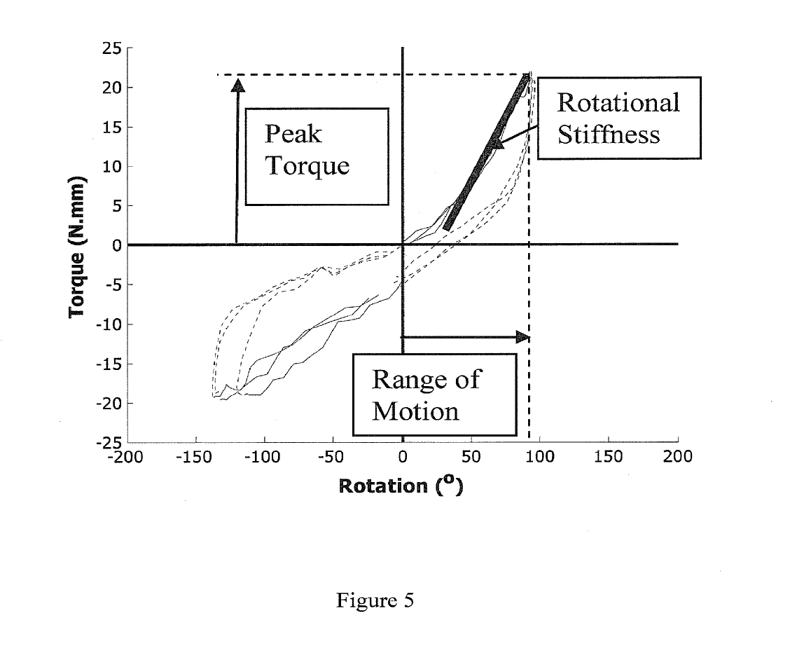

In one embodiment, range of motion is the maximum angular-displacement associated with initial subject discomfort as is done clinically. In another embodiment, the peak torque is the torque associated with the maximum angular displacement. In another embodiment, the rotational stiffness is the slope of the torque angular-displacement curve shown in FIG. 5.

In one embodiment, the range of motion is determined from the maximum angular-displacement associated with initial subject discomfort as the subject's arm is passively rotated. In another embodiment, the device 10 can measure a range of motion of 0-180°, with about 95% accuracy and repeatability.

In another embodiment, the range of motion is tested by using a known angle (as measured with a goniometer). In another embodiment, determination of the range of motion enables measuring the accuracy of the device 10 and the calibration of the device 10.

In another embodiment, the peak torque is the torque associated with the maximum angular-displacement measured. In another embodiment, the torque has about 95% accuracy and repeatability.

In another embodiment, the rotational stiffness is calculated as the slope of the torque angular-displacement curve.

In one embodiment, the device 10 measures passive shoulder mechanics before and at various time points following treatment. In another embodiment, the device 10 is monitors the progress of a subject's rehabilitation following a procedure in which increased shoulder stiffness and decreased range of motion are of concern. In yet another embodiment, the device 10 is assesses the effect of immobilization on repair integrity, shoulder stiffness and range of motion following rotator cuff repair surgery. In another embodiment, the device 10 quantifies shoulder stiffness and range of motion.

In one embodiment, provided herein is a method for making the device 10 for measuring shoulder stiffness and range of motion in a human subject. In another embodiment, the method involves joining an adjustable armature 14 to a portable stand 18. In another embodiment, the armature 14 is secured to the subject's forearm, near the elbow, with Velcro straps. In another embodiment, the method involves resting the stand 18 on locking caster wheels 22, that are, in another embodiment, constructed of any suitable material known in the art, such as, but not limited to extruded aluminum. In another embodiment, the stand is adjusted from about 70 to about 140 cm in height to account for >95% of the adult population. In another embodiment, the method involves mounting a six-degree of freedom load cell 26 below the armature 14 and doing so in-line with a rigid axle 38 which passes through a low-friction roller bearing joint 30. In another embodiment, an orientation sensor 34 is placed on the undersurface of the axle, as well as a handle 50 from which the operator rotates the subject's arm in either internal or external directions. In another embodiment, the operator passively rotates the subject's arm in either internal or external directions. In another embodiment, an orientation sensor 34 is be placed on the undersurface of the axle, as well as a handle 50 from which the operator will rotate the subject's arm in either internal or external directions. In another embodiment, the method involves connecting the the sensors 26, 34 of the device 10 using connecting modules to a processing machine 46, for processing measured data. In another embodiment, the method involves processing the data using the processing machine 46 and a suitable analytic software available in the art, such as, but not limited to, LabView and visualizing the results using a visual display or monitor 54 that, in another embodiment, is mounted on the device 10 for displaying torque and angle displacement measurements. In another embodiment, the processing machine 46 comprises a torque cell amplifier 58. In another embodiment, the processing machine comprises a data acquisition card.

In one embodiment, the device 10 is unique in that it comprasises an armature 14 that adjusts in height. In another embodiment, the device 10 comprises a stop to prevent armature and sensor assembly from falling and hitting the ground. In another embodiment, the armature 14 can be easily removed to switch between left and right. In another embodiment, the armature is made out of soft non-porous material that is easy to clean and disinfect.

The device 10 enables the use of data processing techniques to minimize volitional noise. In another embodiment, the device 10 enables test subjects to be examined either seated or standing. In another embodiment, anesthesia is not required to examine a human subject. In another embodiment, human specific for torques and angles are employed in the examination of a subject using the device 10 provided herein.

In one embodiment, provided herein is a method for accurately quantifying shoulder joint stiffness comprising using the device provided herein.

In another embodiment, provided herein a method of measuring joint stiffness in the shoulder of a human subject comprising the steps of placing the device 10 on either side of said subject's upper extremity and measuring a set of parameters as the upper extremity is externally and internally rotated by an operator. In another embodiment, the upper extremity is rotated passively.

In one embodiment, provided herein is a method of monitoring the progress of a human subject's rehabilitation following a procedure in which increased shoulder stiffness and decreased range of motion are of concern, comprising the step of using the device 10 provided herein.

In another embodiment, provided herein a method of assessing the effect of immobilization on repair integrity, shoulder stiffness and range of motion following rotator cuff repair surgery in a human subject, the method comprising the step of using the device 10 provided herein.

In one embodiment, provided herein is a method of measuring passive shoulder mechanics before and at various pre-determined time points following treatment, the method comprising the step of using the device 10 provided herein. In another embodiment, it is to be understood that a skilled artisan is able to devise a set of time points as required for each particular subject or pool of subjects.

In one embodiment, the term “about” is, in quantitative terms, plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans.

The following examples are presented in order to more fully illustrate the embodiments of the invention. They should in no way be construed, however, as limiting the broad scope of the invention.

### EXAMPLES

### Example 1

### Calculating Stiffness of a Known Object

Springs were stretched a known length and connected to the device 10 to apply a known torque versus the angle. The expected spring stiffness given in the manufacturer's spec to the measured spring stiffness calculated from the torque stiffness were then compared (Table 1).

TABLE 1

| | Expected spring | Calculated spring | Percent |
|---|---|---|---|
| Spring | stiffness | stiffness | error |
| 6″ steel music wire | 0.91 lbs/in | 0.977 (0.670) | 7.44% |
| extension spring | | | |
| 10″ steel music wire | 2.15 lbs/in | 2.28 (1.37) | 5.94% |
| extension spring | | | |

### Example 2

### Measuring Torsional Stiffness

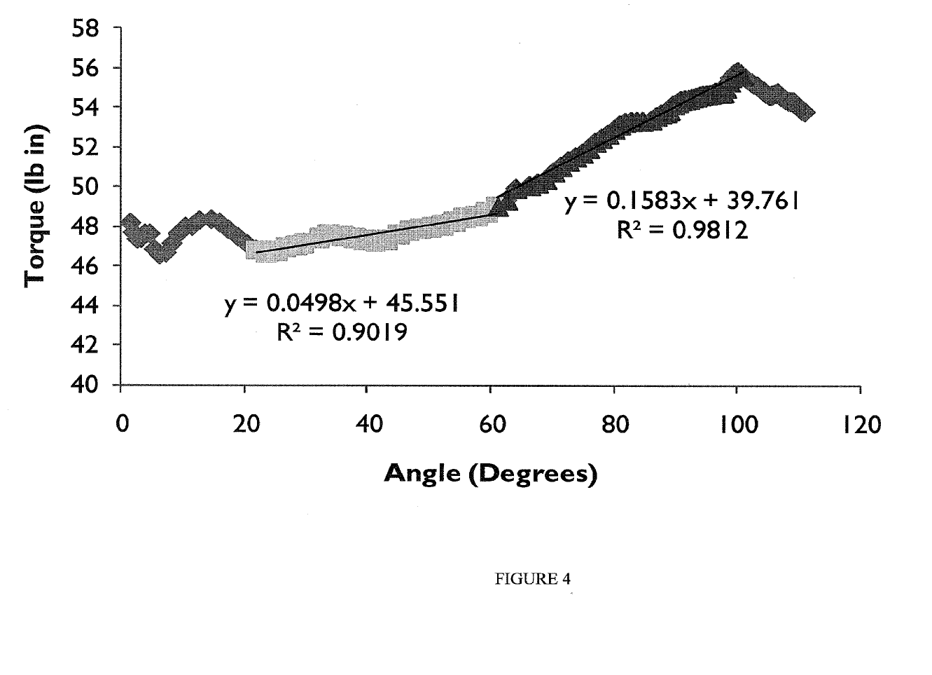

The torsional stiffness of a subject's shoulder in external rotation was found by plotting the torque versus the angle (FIG. 4). Two slopes were used to characterize the torque curve due to its curvilinear nature.

The stiffness was found to be 0.0498 lb in/degree and 0.1583 lb in/degree (FIG. 4).

### Example 3

### Specifications vs Actual Measurements

Table 2 provides data relating to the specifications of the device versus actual measurements taken.

TABLE 2

| | | | Delivered | |
|---|---|---|---|---|
| | | Promised | (Actual | |
| Component | Specification | (Specified) | Performance) | Test Method |
| Sensor | Range of | 0-180° | 0-180° | Measurement |
| Apparatus | motion | | | of range of |
| | (performance) | | | motion |
| | Torque sensor | 95% | <95% | Comparison of |
| | accuracy | | | output |
| | (performance) | | | measurement |
| | | | | to known |
| | | | | torque |
| | Calibration | ≦1 calibration/day | Once per | Visual and |
| | (operational) | | measurement | operational |
| | | | | confirmation |
| | Sensors | Acquire and relay | Acquire and relay | Signal |
| | connectivity | torque and angle | torque and angle | captured in |
| | (operational) | data to processing | data to processing | LabVIEW |
| | | machine | machine | |
| | Materials | Aluminum, foam | Aluminum, foam | Visual |
| | (physical | padding, torque | padding, torque | confirmation |
| | characteristics) | sensor, orientation | sensor, orientation | of materials |
| | | sensor, connecting | sensor, connecting | |
| | | modules, bearing | modules, bearing | |
| | | joint, adjustable | joint, adjustable | |
| | | clamps | clamps | |
| | Durability | withstand 3 ft drop | stop collar to | Measurement |
| | (physical | | prevent drop | of drop visual |
| | characteristics) | | | confirmation |
| Mounting | Materials | Aluminum, plastic | Aluminum, plastic | Visual |
| stand | (physical | wheels with | wheels with | confirmation |
| | characteristics) | brakes | brakes | of materials |
| | Height | ≧12 in | ≧12 in | Measurement |
| | adjustable | | | of maximum |
| | (operational) | | | length |
| | Armature | ≧2 in | ≧2 in | Measurement |
| | length | | | of maximum |
| | adjustable | | | and minimum |
| | (operational) | | | length |

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A device for measuring shoulder stiffness and range of motion in a human subject, the device comprising:
an adjustable armature that is capable of being secured to a forearm of a human subject;
a sensor assembly comprising a torque load cell, ball bearing joints, and an orientation sensor,
wherein said cell is mounted below said armature, in-line with a rigid axle, and capable of passing through said ball bearing joint, wherein said orientation sensor disposed on the undersurface of said axle, and
wherein said device is capable of measuring shoulder stiffness and range of motion during passive rotation of said forearm of said human subject.

2. The device of claim 1, wherein said adjustable armature is adjustable in height.

3. The device of claim 1, wherein said adjustable armature can be easily removed to switch between left and right extremity.

4. The device of claim 1, wherein said adjustable armature is made out of soft non-porous material.

5. The device of claim 1, wherein said human subject is seated or standing.

6. The device of claim 1, further comprising a processing machine to process measured data relating to shoulder stiffness and range of motion.

7. The device of claim 1, wherein the device is capable of measuring torque and angular displacement as the human subject's arm is passively rotated.

8. The device of claim 1, wherein said armature is mounted on a stand.

9. The device of claim 4, wherein said stand rests on locking caster wheels constructed of extruded aluminum and adjusted from about 70 to about 140 cm in height to account for more than about 95% of the adult human population.

10. The device of claim 1, wherein said subject is effectively diagnosed as having unilateral shoulder stiffness and range of motion.

11. The device of claim 1, wherein said subject is a healthy individual.

12. The device if claim 1, wherein said armature is secured near the elbow of said subject's, with Velcro straps.

13. The device of claim 1, wherein said torque load cell mounted below the armature is a six-degree of freedom torque load cell.

14. The device of claim 1, wherein said load cell has a maximum torque of 100 N·mm.

15. The device of claim 1, wherein said ball bearing joint is a low-friction roller bearing joint.

16. The device of claim 1, wherein said rigid axle comprises a handle that enables an operator to rotate the subject's arm in an internal direction or external direction.

17. The device of claim 1, wherein said device is capable of being calibrated to provide accuracy or precision in measuring rotational stiffness.

18. The device of claim 1, wherein said device is capable of measuring a set of parameters, wherein said parameters comprise the number of internal/external cycles and the speed of rotation.

19. The device of claim 18, wherein said parameters enable measuring the range of motion, the peak torque, and the rotational stiffness.

20. The device of claim 19, wherein the range of motion is determined from the maximum angular-displacement associated with initial subject discomfort

21. The device of claim 19, wherein the peak torque is the torque associated with the maximum angular-displacement measured.

22. The device of claim 19, wherein the rotational stiffness is calculated as the slope of the torque angular-displacement curve.

23. The device of claim 1, wherein said subject's arm is fully adducted.

24. The device of claim 1, wherein said device is capable of measuring passive shoulder mechanics before and at various time points following treatment.

25. The device of claim 1, wherein said device is capable of monitoring the progress of a subject's rehabilitation following a procedure in which increased shoulder stiffness and decreased range of motion are of concern.

26. The device of claim 1, wherein said device is capable of assessing the effect of immobilization on repair integrity, shoulder stiffness and range of motion following rotator cuff repair surgery.

27. The device of claim 1, wherein said device is capable of quantifying shoulder stiffness and range of motion.

28. A method for accurately quantifying shoulder joint stiffness, the method comprising the step of using the device of claim 1.

29. A method of measuring joint stiffness in the shoulder of a human subject comprising the steps of placing the device of claim 1 on either side of said subject's upper extremity and measuring a set of parameters as the upper extremity is externally and internally rotated by an operator.

30. The method of claim 29, wherein said upper extremity is rotated passively.

31. A method of monitoring the progress of a human subject's rehabilitation following a procedure in which increased shoulder stiffness and decreased range of motion are of concern, comprising the step of using the device of claim 1.

32. A method of assessing the effect of immobilization on repair integrity, shoulder stiffness and range of motion following rotator cuff repair surgery in a human subject, the method comprising the step of using the device of claim 1.

33. A method of measuring passive shoulder mechanics prior to and at various pre-determined time points following treatment comprising the step of using the device of claim 1.

34. A method of making the device of claim 1.
